(19) United States
(12) Patent Application Publication
(10) Pub. No.: US 2024/0324994 A1
(43) Pub. Date: Oct. 3, 2024

(54) ULTRASOUND DIAGNOSTIC SYSTEM AND CONTROL METHOD OF ULTRASOUND DIAGNOSTIC SYSTEM

(71) Applicant: FUJIFILM Corporation, Tokyo (JP)

(72) Inventor: Ryo SHINTATE, Ashigarakami-gun (JP)

(73) Assignee: FUJIFILM Corporation, Tokyo (JP)

(21) Appl. No.: 18/609,268

(22) Filed: Mar. 19, 2024

(30) Foreign Application Priority Data
Mar. 27, 2023 (JP) 2023-049629

(51) Int. Cl. A61B 8/00

(52) U.S. Cl. CPC A61B 8/4472; A61B 8/4438; A61B 8/461

(57) ABSTRACT

Provided are an ultrasound diagnostic system and a control method of an ultrasound diagnostic system capable of easily discerning a diagnostic apparatus and an ultrasound probe that are wirelessly connected to each other.

There is provided an ultrasound diagnostic system including: at least one ultrasound probe; and at least one diagnostic apparatus that is pairing-settable with the at least one ultrasound probe, in which the at least one ultrasound probe has a probe side identification mark display unit, the at least one diagnostic apparatus has a diagnostic apparatus side identification mark display unit, and the ultrasound probe and the diagnostic apparatus that are mutually pairing-set display a common identification mark on the respective probe side identification mark display unit and diagnostic apparatus side identification mark display unit.

### CROSS-REFERENCE TO RELATED APPLICATIONS

The present application claims priority under 35 U.S.C. § 119 to Japanese Patent Application No. 2023-049629, filed on Mar. 27, 2023. The above application is hereby expressly incorporated by reference, in its entirety, into the present application.

### BACKGROUND OF THE INVENTION

### 1. Field of the Invention

The present invention relates to an ultrasound diagnostic system comprising at least one ultrasound probe and at least one diagnostic apparatus that are mutually pairing-set, and a control method of the ultrasound diagnostic system.

### 2. Description of the Related Art

Hitherto, in the medical field, an ultrasound diagnostic apparatus using ultrasound images has been put into practical use. The ultrasound diagnostic apparatus typically comprises an ultrasound probe incorporating a transducer array, and an apparatus body connected to the ultrasound probe, and transmits an ultrasound beam from the ultrasound probe toward a subject, receives an ultrasound echo from the subject through the ultrasound probe, and electrically processes a reception signal thereof to generate an ultrasound image.

In addition, JP2011-087840A discloses an ultrasound diagnostic apparatus that establishes wireless connection between an ultrasound probe and an apparatus body through wireless communication. The ultrasound image acquired by the ultrasound probe is displayed on a monitor of the apparatus body.

### SUMMARY OF THE INVENTION

In a case in which wireless connection is established between the ultrasound probe and the apparatus body in this way, operability and mobility of the ultrasound probe are improved, and efficient diagnosis can be performed.

However, in a case in which there are a plurality of diagnostic apparatuses and a plurality of ultrasound probes in medical settings such as a hospital, and any of the diagnostic apparatuses is wirelessly connected to any of the ultrasound probes, it takes time and effort to discern the diagnostic apparatus and the ultrasound probe that are wirelessly connected to each other, which imposes a burden on a user who urgently needs to use the ultrasound diagnostic apparatus. In addition, a plurality of ultrasound probes may also be wirelessly connected to one diagnostic apparatus.

Therefore, it takes time to discern the ultrasound probe that is wirelessly connected to the diagnostic apparatus, and there is also a concern about malfunctions such as attempting to use an ultrasound probe that is not wirelessly connected to the diagnostic apparatus.

The present invention has been made in order to solve such a conventional problem, and an object of the present invention is to provide an ultrasound diagnostic system and a control method of an ultrasound diagnostic system capable of easily discerning a diagnostic apparatus and an ultrasound probe that are wirelessly connected to each other.

According to the following configuration, the above-described object can be achieved.

[1] An ultrasound diagnostic system comprising:
at least one ultrasound probe; and
at least one diagnostic apparatus that is pairing-settable with the at least one ultrasound probe,
in which the at least one ultrasound probe has a probe side identification mark display unit,
the at least one diagnostic apparatus has a diagnostic apparatus side identification mark display unit, and
the ultrasound probe and the diagnostic apparatus that are mutually pairing-set display a common identification mark on the respective probe side identification mark display unit and diagnostic apparatus side identification mark display unit.

[2] The ultrasound diagnostic system according to [1],
in which the common identification mark has any of a common color, number, symbol, or figure for the ultrasound probe and diagnostic apparatus that are mutually pairing-set.

The ultrasound diagnostic system according to [1] or [2],
in which at least one of the probe side identification mark display unit or the diagnostic apparatus side identification mark display unit consists of a light emitting unit that emits light of a common color to the ultrasound probe and the diagnostic apparatus that are mutually pairing-set as the common identification mark.

[4] The ultrasound diagnostic system according to any one of [1] to [3],
in which the at least one diagnostic apparatus has a diagnostic apparatus side monitor that displays an ultrasound image generated by using a pairing-set ultrasound probe among the at least one ultrasound probe.

[5] The ultrasound diagnostic system according to [4],
in which the diagnostic apparatus side identification mark display unit consists of the diagnostic apparatus side monitor.

[6] The ultrasound diagnostic system according to [4],
in which the at least one diagnostic apparatus has a probe list creation unit that creates a probe list in which list information of the at least one ultrasound probe is described and which is displayed on the diagnostic apparatus side monitor, and
an ultrasound probe selected from the list information described in the probe list of a certain diagnostic apparatus among the at least one diagnostic apparatus is pairing-set with the certain diagnostic apparatus.

[7] The ultrasound diagnostic system according to any one of [1] to [6],
in which the at least one ultrasound probe has a probe side monitor, and
the probe side identification mark display unit consists of the probe side monitor.

[9] The ultrasound diagnostic system according to [1],
in which, in a case in which a plurality of the ultrasound probes are pairing-set with one diagnostic apparatus among the at least one diagnostic apparatus, the common identification mark is displayed in mutually different forms on the probe side identification mark display units of the plurality of ultrasound probes.

[9] The ultrasound diagnostic system according to [8],
in which the common identification mark is displayed at mutually different positions of the probe side identification mark display units of the plurality of ultrasound probes.

[10] The ultrasound diagnostic system according to [8],
in which the common identification mark is displayed on the plurality of ultrasound probes in a common color and with mutually different numbers, symbols, or figures.

[11] The ultrasound diagnostic system according to any one of [1] to [10],
in which the at least one ultrasound probe has a connection check switch operated by a user, and
in a case in which the connection check switch of a certain ultrasound probe among the at least one ultrasound probe is operated, the common identification mark is displayed on the probe side identification mark display unit of the certain ultrasound probe and the diagnostic apparatus side identification mark display unit of the diagnostic apparatus that is pairing-set with the certain ultrasound probe.

[12] The ultrasound diagnostic system according to any one of [1] to [11],
in which at least one of the mutually pairing-set ultrasound probe or diagnostic apparatus transmits a signal representing the common identification mark displayed on the probe side identification mark display unit and the diagnostic apparatus side identification mark display unit toward surroundings.

[13] A control method of an ultrasound diagnostic system including at least one ultrasound probe and at least one diagnostic apparatus, the control method comprising:
mutually pairing-setting a certain ultrasound probe and a certain diagnostic apparatus among the at least one ultrasound probe and the at least one diagnostic apparatus; and
displaying a common identification mark on a probe side identification mark display unit of the ultrasound probe and a diagnostic apparatus side identification mark display unit of the diagnostic apparatus that are mutually pairing-set.

In the ultrasound diagnostic system according to the present invention, the at least one ultrasound probe has a probe side identification mark display unit, the at least one diagnostic apparatus has a diagnostic apparatus side identification mark display unit, and the ultrasound probe and the diagnostic apparatus that are mutually pairing-set display a common identification mark on the respective probe side identification mark display unit and diagnostic apparatus side identification mark display unit. Therefore, it is possible to easily discern the diagnostic apparatus and the ultrasound probe that are wirelessly connected to each other.

### BRIEF DESCRIPTION OF THE DRAWINGS

FIG. 1 is a block diagram showing a configuration of an ultrasound diagnostic system according to Embodiment 1 of the present invention.

FIG. 2 is a block diagram showing internal configurations of an ultrasound probe and a diagnostic apparatus that are wirelessly connected to each other in the ultrasound diagnostic system according to Embodiment 1 of the present invention.

FIG. 3 is a block diagram showing an internal configuration of a transmission and reception circuit of the ultrasound probe in Embodiment 1 of the present invention.

FIG. 4 is a block diagram showing an internal configuration of an image generation unit of the ultrasound probe in Embodiment 1 of the present invention.

FIG. 5 is a perspective view showing an appearance of the ultrasound probe in Embodiment 1 of the present invention.

FIG. 6 is a diagram showing the diagnostic apparatus in Embodiment 1 of the present invention.

FIG. 7 is a flowchart showing an operation of the ultrasound diagnostic system according to Embodiment 1 of the present invention.

FIG. 8 is a diagram showing the diagnostic apparatus in which an ultrasound image is displayed on a diagnostic apparatus side monitor in Embodiment 1 of the present invention.

FIG. 9 is a block diagram showing an internal configuration of a diagnostic apparatus in Embodiment 2 of the present invention.

FIG. 10 is a diagram showing the diagnostic apparatus in Embodiment 2 of the present invention.

FIG. 11 is a block diagram showing an internal configuration of an ultrasound probe in Embodiment 3 of the present invention.

FIG. 12 is a perspective view showing an appearance of the ultrasound probe in Embodiment 3 of the present invention.

FIG. 13 is a perspective view showing an appearance of an ultrasound probe in Embodiment 4 of the present invention.

FIG. 14 is a diagram showing a display screen of a probe side monitor in Embodiment 5 of the present invention.

FIG. 15 is a diagram showing a display screen of a probe side monitor in a modification example of Embodiment 5 of the present invention.

### DESCRIPTION OF THE PREFERRED EMBODIMENTS

Hereinafter, embodiments of the present invention will be described with reference to the accompanying drawings.

The description of configuration requirements to be described below is made based on a representative embodiment of the present invention, but the present invention is not limited to such an embodiment.

In the present specification, a numerical range represented by “to” means a range including numerical values described before and after “to” as a lower limit value and an upper limit value.

In the present specification, “same” and “identical” include error ranges generally allowed in the technical field.

### Embodiment 1

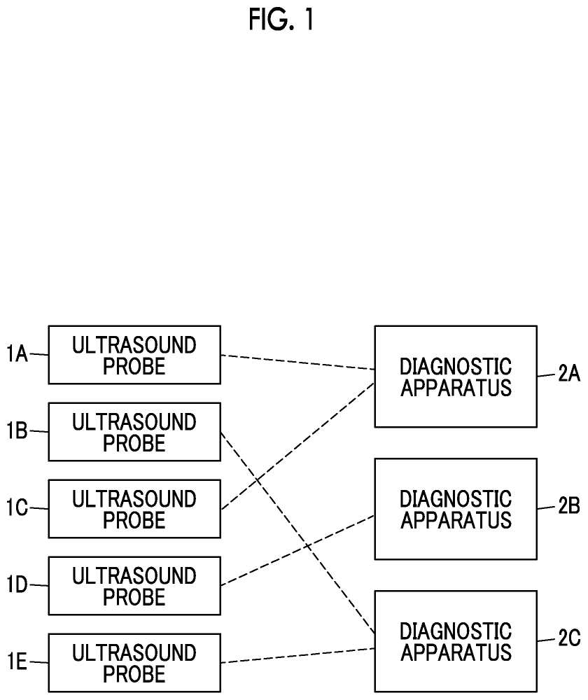

FIG. 1 shows a configuration of an ultrasound diagnostic system according to Embodiment 1 of the present invention. The ultrasound diagnostic system comprises a plurality of ultrasound probes 1A to 1E and a plurality of diagnostic apparatuses 2A to 2C. It is assumed that the plurality of diagnostic apparatuses 2A to 2C are each pairing-settable with at least one ultrasound probe among the plurality of ultrasound probes 1A to 1E.

In addition, FIG. 1 shows a state in which the ultrasound probes 1A and 1C are pairing-set with the diagnostic apparatus 2A, the ultrasound probe 1D is pairing-set with the diagnostic apparatus 2B, and the ultrasound probes 1B and 1E are pairing-set with the diagnostic apparatus 2C.

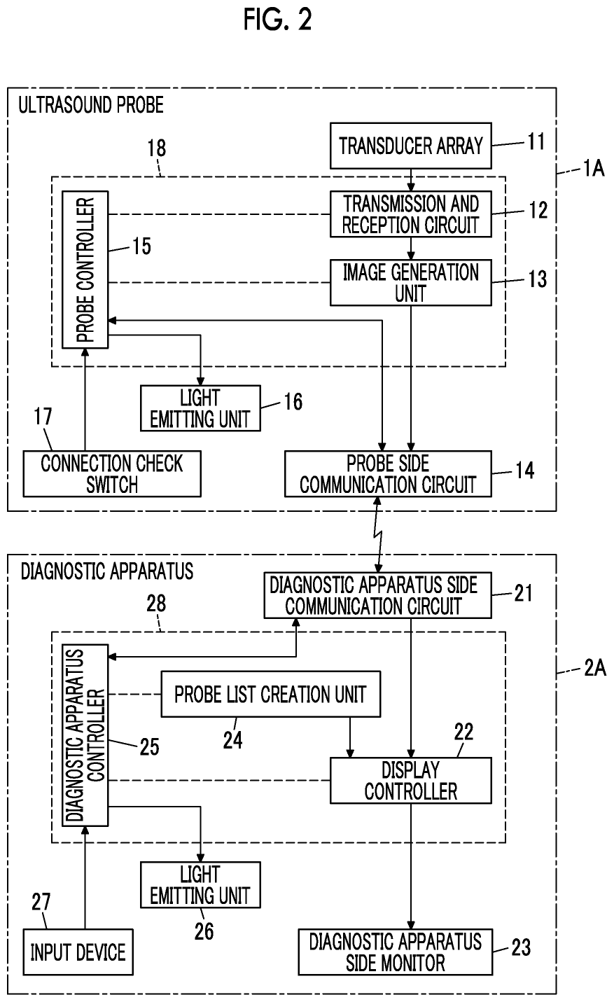

Here, FIG. 2 shows internal configurations of the ultrasound probe 1A and the diagnostic apparatus 2A that are pairing-set and wirelessly connected to each other.

The ultrasound probe 1A has a transducer array 11, and a transmission and reception circuit 12, an image generation unit 13, and a probe side communication circuit 14 are sequentially connected to the transducer array 11, and a probe controller 15 is connected to the transmission and reception circuit 12 and the image generation unit 13.

In addition, the ultrasound probe 1A has a light emitting unit 16 and a connection check switch 17, and the light emitting unit 16 and the connection check switch 17 are each connected to the probe controller 15.

A probe side processor 18 is composed of the transmission and reception circuit 12, the image generation unit 13, and the probe controller 15.

Meanwhile, the diagnostic apparatus 2A has a diagnostic apparatus side communication circuit 21, and a display controller 22 and a diagnostic apparatus side monitor 23 are sequentially connected to the diagnostic apparatus side communication circuit 21. In addition, the diagnostic apparatus 2A has a probe list creation unit 24, and a diagnostic apparatus controller 25 is connected to the display controller 22 and the probe list creation unit 24.

Further, the diagnostic apparatus 2A has a light emitting unit 26 and an input device 27, and the light emitting unit 26 and the input device 27 are each connected to the diagnostic apparatus controller 25.

A diagnostic apparatus side processor 28 is composed of the display controller 22, the probe list creation unit 24, and the diagnostic apparatus controller 25.

The transducer array 11 of the ultrasound probe 1A has a plurality of ultrasound transducers that are one-dimensionally or two-dimensionally arranged. These ultrasound transducers each transmit an ultrasound wave in accordance with a drive signal supplied from the transmission and reception circuit 12, receive an ultrasound echo from a subject, and output a signal based on the ultrasound echo. For example, each ultrasound transducer includes a piezoelectric body and electrodes formed at both ends of the piezoelectric body. The piezoelectric body consists of a piezoelectric ceramic represented by lead zirconate titanate (PZT), a polymer piezoelectric element represented by poly vinylidene di fluoride (PVDF), a piezoelectric single crystal represented by lead magnesium niobate-lead titanate (PMN-PT), or the like.

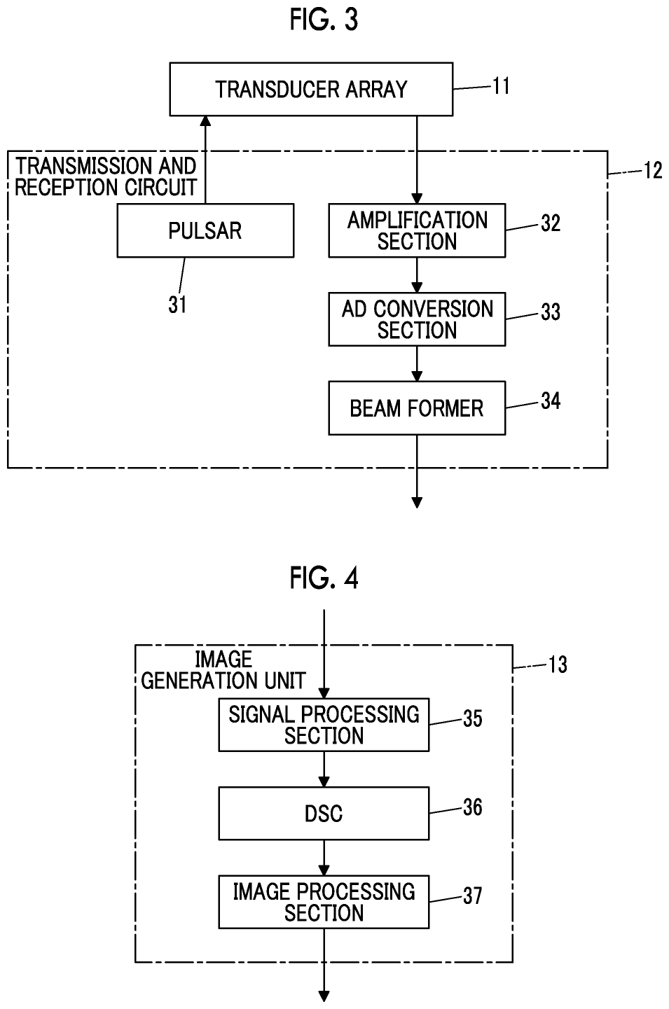

The transmission and reception circuit 12 transmits the ultrasound wave from the transducer array 11 and generates a sound ray signal based on a reception signal acquired by the transducer array 11, under the control of the probe controller 15. As shown in FIG. 3, the transmission and reception circuit 12 has a pulsar 31 connected to the transducer array 11, and an amplification section 32, an analog-to-digital (AD) conversion section 33, and a beam former 34 that are sequentially connected in series to the transducer array 11.

The pulsar 31 includes, for example, a plurality of pulse generators, and adjusts an amount of delay of each of drive signals and supplies the drive signals to the plurality of ultrasound transducers such that ultrasound waves transmitted from the plurality of ultrasound transducers of the transducer array 11 form an ultrasound beam based on a transmission delay pattern selected according to a control signal from the probe controller 15. In this way, in a case in which a pulsed or continuous wave-like voltage is applied to the electrodes of the ultrasound transducer of the transducer array 11, the piezoelectric body expands and contracts to generate a pulsed or continuous wave-like ultrasound wave from each of the ultrasound transducers, whereby an ultrasound beam is formed from the combined wave of these ultrasound waves.

The transmitted ultrasound beam is reflected in, for example, a target such as a site of the subject and propagates toward the transducer array 11 of the ultrasound probe 1A. The ultrasound echo that propagates toward the transducer array 11 in this way is received by each of the ultrasound transducers constituting the transducer array 11. In this case, each of the ultrasound transducers constituting the transducer array 11 receives the propagating ultrasound echo to expand and contract to generate a reception signal, which is an electrical signal, and outputs these reception signals to the amplification section 62.

The amplification section 32 amplifies the signal input from each of the ultrasound transducers constituting the transducer array 11 and transmits the amplified signal to the AD conversion section 33. The AD conversion section 33 converts the signal transmitted from the amplification section 32 into digital reception data. The beam former 34 performs so-called reception focus processing by applying and adding a delay to each reception data received from the AD conversion section 33. Through the reception focus processing, the sound ray signal in which each reception data converted by the AD conversion section 33 is phase-summed and a focus of the ultrasound echo is narrowed down is acquired.

As shown in FIG. 4, the image generation unit 13 has a configuration in which a signal processing section 35, a digital scan converter (DSC) 36, and an image processing section 37 are sequentially connected in series.

The signal processing section 35 generates a B-mode image signal, which is tomographic image information regarding tissues inside the subject, by performing, on the sound ray signal received from the transmission and reception circuit 12, correction of the attenuation due to the distance according to the depth of the reflection position of the ultrasound wave using a sound velocity value set by the probe controller 15 and then performing envelope detection processing.

The DSC 36 converts (raster-converts) the B-mode image signal generated by the signal processing section 35 into an image signal conforming to a normal television signal scanning method.

The image processing section 37 performs various types of necessary image processing such as gradation processing on the B-mode image signal input from the DSC 36 and then sends out the B-mode image signal to the probe side communication circuit 14. Hereinafter, the B-mode image signal that has been subjected to the image processing by the image processing section 37 will be referred to as an ultrasound image.

The probe side communication circuit 14 includes an antenna for transmitting and receiving radio waves, and modulates a carrier based on the ultrasound image generated by the image generation unit 13 to generate a transmission signal representing the ultrasound image, and demodulates a transmission signal received from the diagnostic apparatus 2A. As the modulation method of the carrier, for example, amplitude shift keying (ASK), phase shift keying (PSK), quadrature phase shift keying (QPSK), 16 quadrature amplitude modulation (16QAM), or the like is used.

The probe side communication circuit 14 transmits radio waves from the antenna by supplying the transmission signal generated in this manner to the antenna, and wirelessly transmits the ultrasound image toward the diagnostic apparatus side communication circuit 21 of the diagnostic apparatus 2A.

The probe controller 15 controls each unit of the ultrasound probe 1A based on a control program or the like stored in advance.

The light emitting unit 16 is formed of, for example, a light emitting diode (LED) or an organic light emitting diode (OLED), and constitutes a probe side identification mark display unit that displays an identification mark M1 by emitting light of a common color to the ultrasound probe 1A and the diagnostic apparatus 2A that are mutually pairing-set, under the control of the probe controller 15.

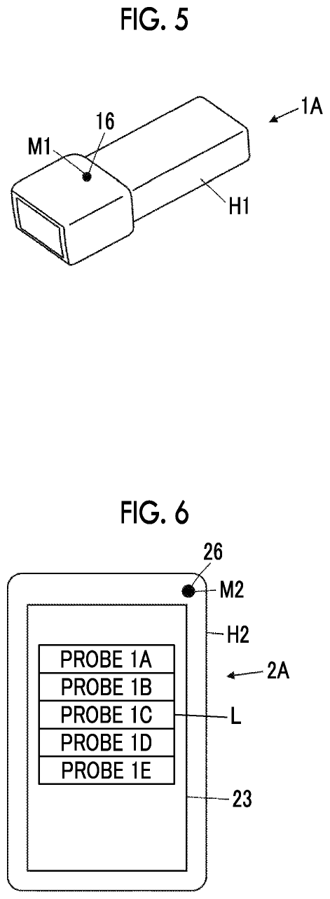

As shown in FIG. 5, the light emitting unit 16 is disposed on an outer surface of a housing H1 of the ultrasound probe 1A or is disposed inside the housing H1 having a light-transmitting property, and the identification mark M1 represented by a color of light emitted from the light emitting unit 16 can be recognized from the outside of the ultrasound probe 1A.

An identification mark M2 represented by a color of light common to the identification mark M1 is also displayed on the diagnostic apparatus 2A that is pairing-set with the ultrasound probe 1A, and the user can identify the ultrasound probe 1A and the diagnostic apparatus 2A that are mutually pairing-set from the fact that the identification mark M1 displayed on the ultrasound probe 1A and the identification mark M2 displayed on the diagnostic apparatus 2A have colors common to each other.

The connection check switch 17 is a switch that is used to check the diagnostic apparatus 2A pairing-set with the ultrasound probe 1A, and in a case in which the connection check switch 17 is operated by the user, the identification mark M1 is displayed by the light emitting unit 16, and the identification mark M2 is displayed on the diagnostic apparatus 2A pairing-set with the ultrasound probe 1A.

Although the probe side processor 18 having the transmission and reception circuit 12, the image generation unit 13, and the probe controller 15 is configured with a central processing unit (CPU) and a control program for causing the CPU to perform various types of processing, the probe side processor 18 may be configured with a field programmable gate array (FPGA), a digital signal processor (DSP), an application specific integrated circuit (ASIC), a graphics processing unit (GPU), or other integrated circuits (ICs) or may be configured with a combination thereof.

In addition, the transmission and reception circuit 12, the image generation unit 13, and the probe controller 15 of the probe side processor 18 can also be configured by being partially or wholly integrated into one CPU or the like.

A battery (not shown) is incorporated into the ultrasound probe 1A, and power is supplied from the battery to each unit of the ultrasound probe 1A.

The diagnostic apparatus side communication circuit 21 of the diagnostic apparatus 2A includes an antenna for transmitting and receiving radio waves, and receives the transmission signal representing the ultrasound image, which is transmitted from the probe side communication circuit 14 of the ultrasound probe 1A, via the antenna and demodulates the received transmission signal, for example, through the method such as ASK, PSK, QPSK, or 16QAM, to send out the ultrasound image to the display controller 22.

The display controller 22 performs predetermined processing on the ultrasound image sent out from the diagnostic apparatus side communication circuit 21 and displays the ultrasound image on the diagnostic apparatus side monitor 23, under the control of the diagnostic apparatus controller 25.

The diagnostic apparatus side monitor 23 is used to display the ultrasound image under the control of the display controller 22, and has, for example, a display device such as a liquid crystal display (LCD) or an organic electroluminescence (EL) display.

Under the control of the diagnostic apparatus controller 25, the probe list creation unit 24 creates a probe list L in which list information of the plurality of ultrasound probes 1A to 1E is described shown in FIG. 6, and displays the created probe list L on the diagnostic apparatus side monitor 23 via the display controller 22.

In a case in which one ultrasound probe is selected by the user from the list information of the plurality of ultrasound probes 1A to 1E described in the probe list L displayed on the diagnostic apparatus side monitor 23 of the diagnostic apparatus 2A, the diagnostic apparatus 2A and the selected ultrasound probe are controlled by the diagnostic apparatus controller 25 such that the selected ultrasound probe is pairing-set with the diagnostic apparatus 2A.

The list information in the probe list L can include not only the names of the plurality of ultrasound probes 1A to 1E provided in the ultrasound diagnostic system but also the types of ultrasound probes such as a linear type, a convex type, and a sector type.

The diagnostic apparatus controller 25 controls each unit of the diagnostic apparatus 2A based on a control program or the like stored in advance.

The light emitting unit 26 is formed of, for example, an LED or an OLED and constitutes a diagnostic apparatus side identification mark display unit that displays the identification mark M2 by emitting light of a common color to the ultrasound probe 1A and the diagnostic apparatus 2A that are mutually pairing-set, under the control of the diagnostic apparatus controller 25.

As shown in FIG. 6, the light emitting unit 26 is disposed on an outer surface of a housing H2 of the diagnostic apparatus 2A or is disposed inside the housing H2 having a light-transmitting property, and the identification mark M2 represented by a color of light emitted from the light emitting unit 26 can be recognized from the outside of the diagnostic apparatus 2A.

The input device 27 is a device that is used for the user to perform an input operation, and includes, for example, a device such as a keyboard, a mouse, a track ball, a touch pad, and a touch sensor that is disposed by being overlaid on the diagnostic apparatus side monitor 23.

The diagnostic apparatus side processor 28 having the display controller 22, the probe list creation unit 24, and the diagnostic apparatus controller 25 is configured with a CPU and a control program for causing the CPU to perform various types of processing, but the diagnostic apparatus side processor 28 may be configured with FPGA, DSP, ASIC, GPU, or other ICs or may be configured with a combination thereof.

In addition, the display controller 22, the probe list creation unit 24, and the diagnostic apparatus controller 25 of the diagnostic apparatus side processor 28 can also be configured by being partially or wholly integrated into one CPU or the like.

In a case in which the diagnostic apparatus 2A is a portable apparatus such as a so-called tablet as shown in FIG. 6, a battery (not shown) is incorporated into the diagnostic apparatus 2A, and power is supplied from the battery to each unit of the diagnostic apparatus 2A.

In addition, although the internal configurations of the ultrasound probe 1A and the diagnostic apparatus 2A among the plurality of ultrasound probes 1A to 1E and the plurality of diagnostic apparatuses 2A to 2C have been described, the other ultrasound probes 1B to 1E have also the same configurations as those of the ultrasound probe 1A, and the other diagnostic apparatuses 2B and 2C also have the same configurations as those of the diagnostic apparatus 2A.

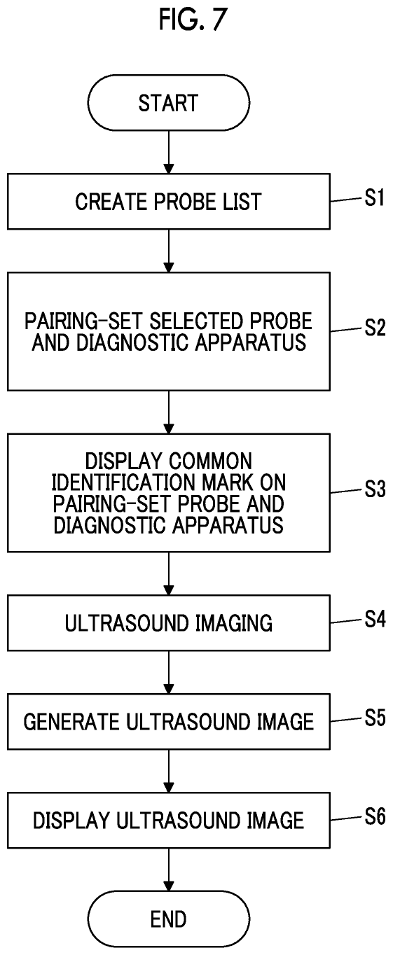

Next, an operation of the ultrasound diagnostic system according to Embodiment 1 will be described with reference to a flowchart shown in FIG. 7.

First, in step S1, the probe list L in which the list information of the plurality of ultrasound probes 1A to 1E is described is created by the probe list creation unit 24 of a certain diagnostic apparatus, and as shown in FIG. 6, the probe list L is displayed on the diagnostic apparatus side monitor 23.

Next, in step S2, in a case in which one ultrasound probe is selected by the user from the probe list L displayed on the diagnostic apparatus side monitor 23, the diagnostic apparatus and the selected ultrasound probe are controlled by the diagnostic apparatus controller 25, and the selected ultrasound probe and the diagnostic apparatus are mutually pairing-set.

Here, for example, it is assumed that the ultrasound probe 1A and the diagnostic apparatus 2A are mutually pairing-set.

In a case in which the ultrasound probe 1A and the diagnostic apparatus 2A are mutually pairing-set in this manner, in step S3, the identification mark M1 is displayed in the color of the light emitted from the light emitting unit 16 of the ultrasound probe 1A, and the identification mark M2 is displayed in the color of the light emitted from the light emitting unit 26 of the diagnostic apparatus 2A.

Here, the identification mark M1 and the identification mark M2 have a common color. It should be noted that the “common color” includes not only the same color but also colors that have spectra similar to each other and that can be perceived by the user as being of the same hue.

Since the identification marks M1 and M2 having a common color are displayed by the light emitting unit 16 of the ultrasound probe 1A and the light emitting unit 26 of the diagnostic apparatus 2A, respectively, the user can check the identification marks M1 and M2 to intuitively and easily discern that the ultrasound probe 1A and the diagnostic apparatus 2A are mutually pairing-set among the plurality of ultrasound probes 1A to 1E and the plurality of diagnostic apparatuses 2A to 2C.

Therefore, in step S4, the user can perform ultrasound imaging on the subject by using the ultrasound probe 1A and the diagnostic apparatus 2A that are mutually pairing-set.

In the ultrasound imaging, the ultrasound probe 1A is put into a contact state with the body surface of the subject, and under the control of the probe controller 15, the transmission of ultrasound waves is started from the plurality of transducers of the transducer array 11 in accordance with the drive signal from the pulsar 31 of the transmission and reception circuit 12, the ultrasound echoes from the internal tissues of the subject are received by the plurality of transducers of the transducer array 11, and the reception signal, which is an analog signal, is output to the amplification section 32 of the transmission and reception circuit 12 and amplified, and converted into a digital signal by the AD conversion section 33.

The reception focus processing is performed by the beam former 34 on the digital signal converted by the AD conversion section 33, the sound ray signal generated through this is sent out to the image generation unit 13, and the ultrasound image representing tomographic image information of the subject is generated by the image generation unit 13 in step S5.

In this case, the sound ray signal is subjected to the attenuation correction corresponding to the depth of the reflection position of the ultrasound wave and the envelope detection processing by the signal processing section 35 of the image generation unit 13, is converted into the image signal conforming to a normal television signal scanning method by the DSC 36, and is subjected to various types of necessary image processing, such as gradation processing, by the image processing section 37.

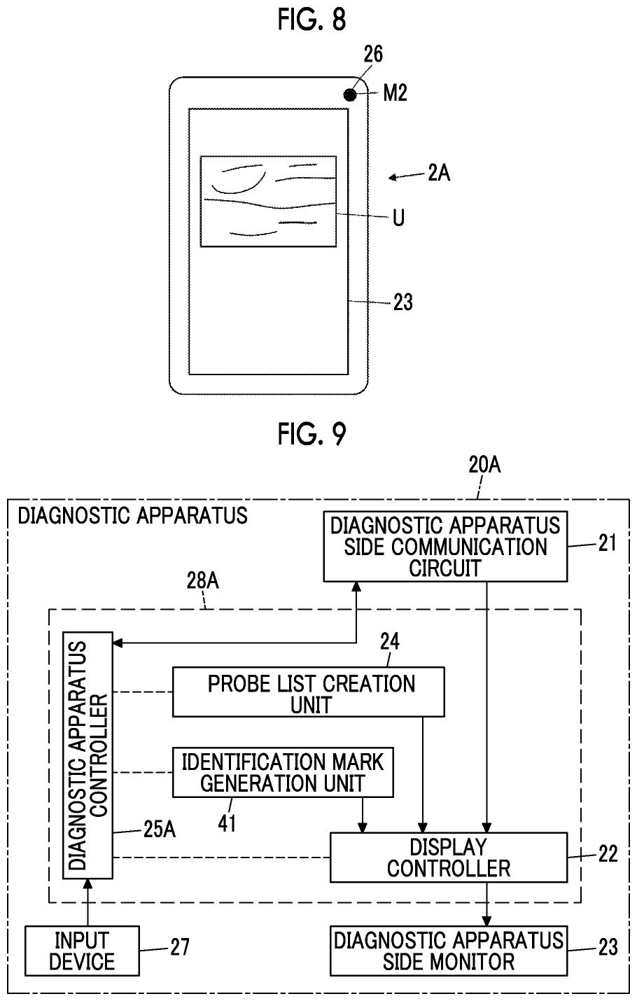

The ultrasound image generated by the image generation unit 13 is transmitted from the probe side communication circuit 14 of the ultrasound probe 1A toward the diagnostic apparatus 2A, is received by the diagnostic apparatus side communication circuit 21 of the diagnostic apparatus 2A, and is then sent out to the diagnostic apparatus side monitor 23 via the display controller 22. In this manner, in step S6, as shown in FIG. 8, an ultrasound image U is displayed on the diagnostic apparatus side monitor 23 of the diagnostic apparatus 2A.

Consequently, a series of operations shown in FIG. 7 ends. Further, in a case in which scanning is performed along the body surface of the subject by using the ultrasound probe 1A, and ultrasound imaging is continued, steps S4 to S6 are repeated.

The identification marks M1 and M2 can be always displayed by the light emitting unit 16 of the ultrasound probe 1A and the light emitting unit 26 of the diagnostic apparatus 2A, respectively, in a case in which the ultrasound probe 1A and the diagnostic apparatus 2A are mutually pairing-set.

Alternatively, the identification marks M1 and M2 can also be displayed by emitting rays of light from the light emitting units 16 and 26 only for a predetermined time from a point in time when the ultrasound probe 1A and the diagnostic apparatus 2A are mutually pairing-set. In this case, it is no longer possible to check the identification marks M1 and M2 when the predetermined time has elapsed from the pairing-setting, but the identification mark M1 is displayed by the light emitting unit 16 of the ultrasound probe 1A and the identification mark M2 is displayed by the light emitting unit 26 of the diagnostic apparatus 2A in response to the operation by the user on the connection check switch 17 of the ultrasound probe 1A. As a result, it is possible to recognize the ultrasound probe 1A and the diagnostic apparatus 2A that are pairing-set and wirelessly connected to each other.

In step S2 of FIG. 7, in a case in which one ultrasound probe is selected by the user from the probe list L displayed on the diagnostic apparatus side monitor 23, for example, highlighting the ultrasound probe selected within the probe list L makes it possible to recognize the pairing-set ultrasound probe 1A from a diagnostic apparatus 2A side by checking the probe list L.

In addition, a configuration may be employed in which the diagnostic apparatus 2A is also equipped with a connection check switch similar to the connection check switch 17 of the ultrasound probe 1A, and in response to the operation on the connection check switch, the identification marks M1 and M2 are displayed on the ultrasound probe 1A and the diagnostic apparatus 2A that are mutually pairing-set, respectively.

Further, in a case in which the ultrasound probe 1A and the diagnostic apparatus 2A are mutually pairing-set, a signal representing the identification mark M1 or M2 can be transmitted from at least one of the ultrasound probe 1A or the diagnostic apparatus 2A toward the surroundings. In addition, this transmission may be repeatedly performed at predetermined time intervals, similarly to a so-called beacon of wireless communication.

For example, a signal representing the color of light to be the identification mark M1 is transmitted from the probe side communication circuit 14 under the control of the probe controller 15 of the ultrasound probe 1A. Consequently, identification marks having colors of rays of light different from that of the identification mark M1, which is used by the ultrasound probe 1A, can be displayed on the ultrasound probe and the diagnostic apparatus in a case in which the ultrasound probes 1B to 1E and the diagnostic apparatuses 2B and 2C that are not yet pairing-set are newly pairing-set.

Similarly, a signal representing the color of light to be the identification mark M2 may be transmitted from the diagnostic apparatus side communication circuit 21 under the control of the diagnostic apparatus controller 25 of the diagnostic apparatus 2A. Consequently, identification marks having colors of rays of light different from that of the identification mark M2, which is used by the diagnostic apparatus 2A, can be displayed on the ultrasound probe and the diagnostic apparatus to be newly pairing-set.

In addition, both the transmission of the signal representing the color of light to be the identification mark M1 from the ultrasound probe 1A and the transmission of the signal representing the color of light to be the identification mark M2 from the diagnostic apparatus 2A may be performed. As a result, identification marks different from the common identification marks M1 and M2, which are used by the ultrasound probe 1A and the diagnostic apparatus 2A, can be displayed on the ultrasound probe and the diagnostic apparatus to be newly pairing-set.

### Embodiment 2

In Embodiment 1 described above, in the diagnostic apparatus 2A, the diagnostic apparatus side identification mark display unit that displays the identification mark M2 is composed of the light emitting unit 26 formed of an LED or an OLED, but the present invention is not limited to this.

FIG. 9 shows an internal configuration of a diagnostic apparatus 20A in Embodiment 2. The diagnostic apparatus 20A comprises an identification mark generation unit 41 instead of the light emitting unit 26 and uses a diagnostic apparatus controller 25A and a diagnostic apparatus side processor 28A instead of the diagnostic apparatus controller 25 and the diagnostic apparatus side processor 28, with respect to the diagnostic apparatus 2A shown in FIG. 2, and other configurations are the same as those of the diagnostic apparatus 2A.

The identification mark generation unit 41 is connected to the diagnostic apparatus controller 25A, and the display controller 22 is connected to the identification mark generation unit 41.

The diagnostic apparatus side processor 28A is composed of the display controller 22, the probe list creation unit 24, the identification mark generation unit 41, and the diagnostic apparatus controller 25A.

The identification mark generation unit 41 generates the identification mark M2 having a color of light common to the identification mark M1 of the ultrasound probe 1A under the control of the diagnostic apparatus controller 25A.

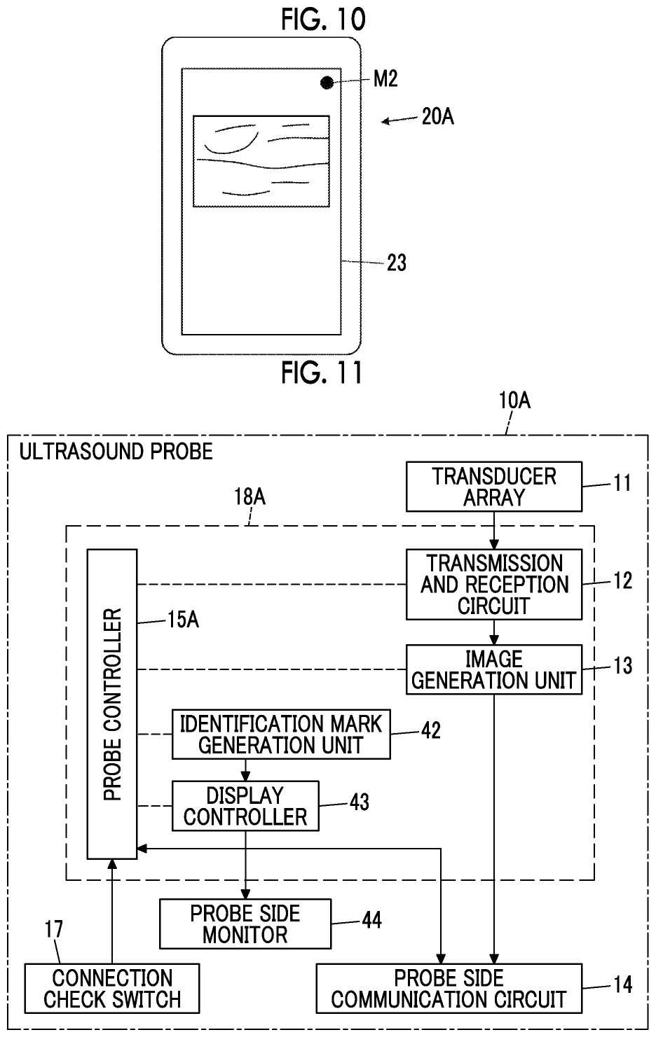

The identification mark M2 generated by the identification mark generation unit 41 is sent out to the diagnostic apparatus side monitor 23 via the display controller 22 and is displayed at a predetermined position of the diagnostic apparatus side monitor 23 as shown in FIG. 10.

That is, in the diagnostic apparatus 20A, the diagnostic apparatus side identification mark display unit that displays the identification mark M2 is composed of the diagnostic apparatus side monitor 23.

As described above, even in a case in which the diagnostic apparatus side monitor 23 is made to function as the diagnostic apparatus side identification mark display unit, and the identification mark M2 is displayed on the diagnostic apparatus side monitor 23, it is possible to easily discern the ultrasound probe 1A and the diagnostic apparatus 2A that are pairing-set and wirelessly connected to each other, in the same manner as in Embodiment 1.

### Embodiment 3

In Embodiment 1 described above, in the ultrasound probe 1A, the probe side identification mark display unit that displays the identification mark M1 is composed of the light emitting unit 16 formed of an LED or an OLED, but the present invention is not limited to this.

FIG. 11 shows an internal configuration of an ultrasound probe 10A in Embodiment 3. The ultrasound probe 10A comprises an identification mark generation unit 42, a display controller 43, and a probe side monitor 44 instead of the light emitting unit 16, and uses a probe controller 15A and a probe side processor 18A instead of the probe controller 15 and the probe side processor 18, with respect to the ultrasound probe 1A shown in FIG. 2, and other configurations are the same as those of the ultrasound probe 1A.

The probe side monitor 44 is connected to the identification mark generation unit 42 via the display controller 43, and the identification mark generation unit 42 and the display controller 43 are connected to the probe controller 15A.

The probe side processor 18A is composed of the transmission and reception circuit 12, the image generation unit 13, the identification mark generation unit 42, the display controller 43, and the probe controller 15A.

The identification mark generation unit 42 generates the identification mark M1 under the control of the probe controller 15A.

The display controller 43 performs predetermined processing on the identification mark M1 generated by the identification mark generation unit 42 and displays the identification mark M1 on the probe side monitor 44, under the control of the probe controller 15A.

The probe side monitor 44 is used to display the identification mark M1 under the control of the display controller 43 and has a display device such as an LCD or an organic EL display, for example.

The identification mark M1 generated by the identification mark generation unit 42 is displayed on the probe side monitor 44 via the display controller 43.

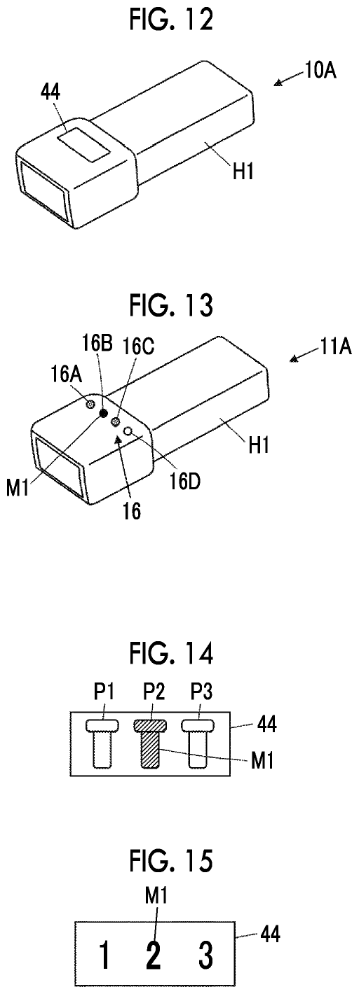

As shown in FIG. 12, the probe side monitor 44 is disposed on the outer surface of a housing H1 of the ultrasound probe 10A, and the identification mark M1 displayed on the probe side monitor 44 can be recognized from the outside of the ultrasound probe 10A.

That is, in the ultrasound probe 10A, the probe side identification mark display unit that displays the identification mark M1 is composed of the probe side monitor 44.

For example, by using the ultrasound probe 10A in combination with the diagnostic apparatus 2A in Embodiment 1 and displaying the identification mark M1 represented by the color of light common to the identification mark M2 of the diagnostic apparatus 2A on the probe side monitor 44 of the ultrasound probe 10A, it is possible to easily discern the ultrasound probe 10A and the diagnostic apparatus 2A that are pairing-set and wirelessly connected to each other, as in the same manner as in Embodiment 1.

In addition, by using the ultrasound probe 10A in combination with the diagnostic apparatus 20A in Embodiment 2, the identification marks M1 and M2 having any of a common color, number, symbol, or figure can also be displayed on the probe side monitor 44 of the ultrasound probe 10A and the diagnostic apparatus side monitor 23 of the diagnostic apparatus 20A, respectively. Even in this manner, by checking the identification mark M1 of the ultrasound probe 10A and the identification mark M2 of the diagnostic apparatus 20A, it is possible to easily discern the ultrasound probe 10A and the diagnostic apparatus 20A that are pairing-set and wirelessly connected to each other.

### Embodiment 4

FIG. 13 shows an appearance of an ultrasound probe 11A in Embodiment 4. The ultrasound probe 11A has, as the light emitting unit 16, four LEDs 16A to 16D arranged on the outer surface of the housing H1 of the ultrasound probe 11A, and the other configurations are the same as those of the ultrasound probe 1A in Embodiment 1.

By using the ultrasound probe 11A in combination with the diagnostic apparatus 2A in Embodiment 1 and emitting the color of light common to the identification mark M2 of the diagnostic apparatus 2A from any LED or a plurality of LEDs of the four LEDs 16A to 16D of the ultrasound probe 11A to display the identification mark M1, it is possible to easily discern the ultrasound probe 11A and the diagnostic apparatus 2A that are pairing-set and wirelessly connected to each other, as in the same manner as in Embodiment 1.

In addition, in a case in which a plurality of ultrasound probes including the ultrasound probe 11A are pairing-set with the diagnostic apparatus 2A, the plurality of pairing-set ultrasound probes can be shown by using the four LEDs 16A to 16D.

For example, in FIG. 13, a state is shown in which three LEDs 16A to 16C, among the four LEDs 16A to 16D of the ultrasound probe 11A, are turned on, and the LED 16B, among the three LEDs 16A to 16C, emits light of a color different from that of the remaining LEDs 16A and 16C, or emits light stronger than that of the remaining LEDs 16A and 16C. The identification mark M1 on the ultrasound probe 11A is displayed by the LED 16B.

By checking the three LEDs 16A to 16C that are turned on to recognize that the three ultrasound probes are pairing-set with the diagnostic apparatus 2A and further checking that the light emitted from the LED 16B has a color common to the identification mark M2 of the diagnostic apparatus 2A, the user can recognize that the ultrasound probe 11A is the second ultrasound probe among the three ultrasound probes pairing-set with the diagnostic apparatus 2A.

In the ultrasound probes other than the ultrasound probe 11A among the three ultrasound probes pairing-set with the diagnostic apparatus 2A, the identification mark M1 is displayed by the LED 16A or 16C.

As described above, by displaying the identification marks M1 at mutually different positions of the light emitting unit 16 even in a case in which the identification marks M1 are displayed in the same color of light from the LEDs 16A to 16C of the three ultrasound probes that are pairing-set with the diagnostic apparatus 2A, it is possible to differentiate the ultrasound probes.

Four OLEDs can also be used as the light emitting unit 16 instead of the four LEDs 16A to 16D.

Further, the number of LEDs 16A to 16D need only be plural and is not limited to four.

### Embodiment 5

In the ultrasound probe 10A in Embodiment 3 described above, since the identification mark M1 is displayed on the probe side monitor 44, the plurality of pairing-set ultrasound probes can be displayed on the probe side monitor 44 in a case in which the plurality of ultrasound probes including the ultrasound probe 10A are pairing-set with the diagnostic apparatus 2A or 20A.

For example, a state is shown in which three probe marks P1 to P3 representing three ultrasound probes are displayed on the probe side monitor 44 shown in FIG. 14, and among the three probe marks P1 to P3, the second probe mark P2 is displayed in a color common to the identification mark M2 of the diagnostic apparatus 2A or 20A. The identification mark M1 on the ultrasound probe 10A is displayed by the probe mark P2.

By checking that the three probe marks P1 to P3 are displayed on the probe side monitor 44 to recognize that the three ultrasound probes are pairing-set with the diagnostic apparatus 2A or 20A and further checking that the color of the probe mark P2 is a color common to the identification mark M2 of the diagnostic apparatus 2A or 20A, the user can recognize that the ultrasound probe 10A is the second ultrasound probe among the three ultrasound probes pairing-set with the diagnostic apparatus 2A or 20A.

In the ultrasound probes other than the ultrasound probe 10A among the three ultrasound probes pairing-set with the diagnostic apparatus 2A or 20A, the probe mark P1 or P3 is displayed in a color common to the identification mark M2 of the diagnostic apparatus 2A or 20A, thereby displaying the identification mark M1.

As described above, by displaying the identification marks M1 at mutually different positions of the probe side monitor 44 even in a case in which the identification marks M1 are displayed in the same color of light on the probe side monitors 44 of the three ultrasound probes that are pairing-set with the diagnostic apparatus 2A or 20A, it is possible to differentiate the ultrasound probes.

In addition, a state is shown in which three numbers “1”, “2”, and “3” representing three ultrasound probes are displayed on the probe side monitor 44 shown in FIG. 15 and the second number “2” among the three numbers is displayed in a color common to the identification mark M2 of the diagnostic apparatus 2A or 20A. The identification mark M1 on the ultrasound probe 10A is displayed by the number “2”.

By checking that the three numbers are displayed on the probe side monitor 44 to recognize that the three ultrasound probes are pairing-set with the diagnostic apparatus 2A or 20A and further checking that the color of the second number “2” among the three numbers is a color common to the identification mark M2 of the diagnostic apparatus 2A or 20A, the user can recognize that the ultrasound probe 10A is the second ultrasound probe among the three ultrasound probes pairing-set with the diagnostic apparatus 2A or 20A.

In the ultrasound probes other than the ultrasound probe 10A among the three ultrasound probes pairing-set with the diagnostic apparatus 2A or 20A, the number “1” or “3” is displayed in a color common to the identification mark M2 of the diagnostic apparatus 2A or 20A, thereby displaying the identification mark M1.

As described above, by displaying the identification marks M1 using mutually different numbers even in a case in which the identification marks M1 are displayed in the same color of light on the probe side monitors 44 of the three ultrasound probes that are pairing-set with the diagnostic apparatus 2A or 20A, it is possible to differentiate the ultrasound probes.

In FIG. 15, although the identification marks M1 are displayed by mutually different numbers, the identification mark M1 is not limited to the numbers, and identification marks M1 can also be displayed using mutually different symbols or mutually different figures.

In Embodiments 1 to 5 described above, the ultrasound probes 1A and 10A have the image generation unit 13, but the present invention is not limited to this, and a configuration can also be employed in which the diagnostic apparatuses 2A and 20A have the image generation unit 13.

In addition, the diagnostic apparatuses 2A and 20A are not limited to portable apparatuses as shown in FIG. 6 and may be stationary apparatuses or so-called cart-type diagnostic apparatuses.

Further, FIG. 1 shows the ultrasound diagnostic system comprising the five ultrasound probes 1A to 1E and the three diagnostic apparatuses 2A to 2C, but the present invention is not limited to this, and the present invention can be applied to an ultrasound diagnostic system comprising at least one ultrasound probe and at least one diagnostic apparatus.

### EXPLANATION OF REFERENCES

1A to 1E, 10A, 11A: ultrasound probe
2A to 2C, 20A: diagnostic apparatus
11: transducer array
12: transmission and reception circuit
13: image generation unit
14: probe side communication circuit
15, 15A: probe controller
16, 26: light emitting unit
16A to 16D: LED
17: connection check switch
18, 18A: probe side processor
21: diagnostic apparatus side communication circuit
22, 43: display controller
23: diagnostic apparatus side monitor
24: probe list creation unit
25, 25A: diagnostic apparatus controller
27: input device
28, 28A: diagnostic apparatus side processor
31: pulsar
32: amplification section
33: AD conversion section
34: beam former
35: signal processing section
36: DSC
37: image processing section
41, 42: identification mark generation unit
44: probe side monitor
M1, M2: identification mark
H1, H2: housing
L: probe list
P1 to P3: probe mark

What is claimed is:

1. An ultrasound diagnostic system comprising:
at least one ultrasound probe; and
at least one diagnostic apparatus that is pairing-settable with the at least one ultrasound probe,
wherein the at least one ultrasound probe has a probe side identification mark display unit,
the at least one diagnostic apparatus has a diagnostic apparatus side identification mark display unit, and
the ultrasound probe and the diagnostic apparatus that are mutually pairing-set display a common identification mark on the respective probe side identification mark display unit and diagnostic apparatus side identification mark display unit.

2. The ultrasound diagnostic system according to claim 1,
wherein the common identification mark has any of a common color, number, symbol, or figure for the ultrasound probe and diagnostic apparatus that are mutually pairing-set.

3. The ultrasound diagnostic system according to claim 1,
wherein at least one of the probe side identification mark display unit or the diagnostic apparatus side identification mark display unit consists of a light emitting device that emits light of a common color to the ultrasound probe and the diagnostic apparatus that are mutually pairing-set as the common identification mark.

4. The ultrasound diagnostic system according to claim 1,
wherein the at least one diagnostic apparatus has a diagnostic apparatus side monitor that displays an ultrasound image generated by using a pairing-set ultrasound probe among the at least one ultrasound probe.

5. The ultrasound diagnostic system according to claim 2,
wherein the at least one diagnostic apparatus has a diagnostic apparatus side monitor that displays an ultrasound image generated by using a pairing-set ultrasound probe among the at least one ultrasound probe.

6. The ultrasound diagnostic system according to claim 3,
wherein the at least one diagnostic apparatus has a diagnostic apparatus side monitor that displays an ultrasound image generated by using a pairing-set ultrasound probe among the at least one ultrasound probe.

7. The ultrasound diagnostic system according to claim 4,
wherein the diagnostic apparatus side identification mark display unit consists of the diagnostic apparatus side monitor.

8. The ultrasound diagnostic system according to claim 5,
wherein the diagnostic apparatus side identification mark display unit consists of the diagnostic apparatus side monitor.

9. The ultrasound diagnostic system according to claim 4,
wherein the at least one diagnostic apparatus has a diagnostic apparatus side processor that creates a probe list in which list information of the at least one ultrasound probe is described and which is displayed on the diagnostic apparatus side monitor, and
an ultrasound probe selected from the list information described in the probe list of a certain diagnostic apparatus among the at least one diagnostic apparatus is pairing-set with the certain diagnostic apparatus.

10. The ultrasound diagnostic system according to claim 5,
wherein the at least one diagnostic apparatus has a diagnostic apparatus side processor that creates a probe list in which list information of the at least one ultrasound probe is described and which is displayed on the diagnostic apparatus side monitor, and
an ultrasound probe selected from the list information described in the probe list of a certain diagnostic apparatus among the at least one diagnostic apparatus is pairing-set with the certain diagnostic apparatus.

11. The ultrasound diagnostic system according to claim 1,
wherein the at least one ultrasound probe has a probe side monitor, and
the probe side identification mark display unit consists of the probe side monitor.

12. The ultrasound diagnostic system according to claim 2,
wherein the at least one ultrasound probe has a probe side monitor, and
the probe side identification mark display unit consists of the probe side monitor.

13. The ultrasound diagnostic system according to claim 1,
wherein, in a case in which a plurality of the ultrasound probes are pairing-set with one diagnostic apparatus among the at least one diagnostic apparatus, the common identification mark is displayed in mutually different forms on the probe side identification mark display units of the plurality of ultrasound probes.

14. The ultrasound diagnostic system according to claim 13,
wherein the common identification mark is displayed at mutually different positions of the probe side identification mark display units of the plurality of ultrasound probes.

15. The ultrasound diagnostic system according to claim 13,
wherein the common identification mark is displayed on the plurality of ultrasound probes in a common color and with mutually different numbers, symbols, or figures.

16. The ultrasound diagnostic system according to claim 1,
wherein the at least one ultrasound probe has a connection check switch operated by a user, and
in a case in which the connection check switch of a certain ultrasound probe among the at least one ultrasound probe is operated, the common identification mark is displayed on the probe side identification mark display unit of the certain ultrasound probe and the diagnostic apparatus side identification mark display unit of the diagnostic apparatus that is pairing-set with the certain ultrasound probe.

17. The ultrasound diagnostic system according to claim 2,
wherein the at least one ultrasound probe has a connection check switch operated by a user, and
in a case in which the connection check switch of a certain ultrasound probe among the at least one ultrasound probe is operated, the common identification mark is displayed on the probe side identification mark display unit of the certain ultrasound probe and the diagnostic apparatus side identification mark display unit of the diagnostic apparatus that is pairing-set with the certain ultrasound probe.

18. The ultrasound diagnostic system according to claim 1,
wherein at least one of the mutually pairing-set ultrasound probe or diagnostic apparatus transmits a signal representing the common identification mark displayed on the probe side identification mark display unit and the diagnostic apparatus side identification mark display unit toward surroundings.

19. The ultrasound diagnostic system according to claim 2,
wherein at least one of the mutually pairing-set ultrasound probe or diagnostic apparatus transmits a signal representing the common identification mark displayed on the probe side identification mark display unit and the diagnostic apparatus side identification mark display unit toward surroundings.

20. A control method of an ultrasound diagnostic system including at least one ultrasound probe and at least one diagnostic apparatus, the control method comprising:
mutually pairing-setting a certain ultrasound probe and a certain diagnostic apparatus among the at least one ultrasound probe and the at least one diagnostic apparatus; and
displaying a common identification mark on a probe side identification mark display unit of the ultrasound probe and a diagnostic apparatus side identification mark display unit of the diagnostic apparatus that are mutually pairing-set.
